SEQUENCE LISTING (30 sequences)

SEQ ID NO: 1 - DNA, 22 bp, Artificial Sequence
  synthetic house-keeping gene transcript glyceraldehyde-3-phosphate dehydrogenase (GAPDH) RT-PCR primer
tcaccagggctgcttttaactc

SEQ ID NO: 2 - DNA, 22 bp, Artificial Sequence
  synthetic house-keeping gene transcript glyceraldehyde-3-phosphate dehydrogenase (GAPDH) RT-PCR primer
atgacaagcttcccgttctcag

SEQ ID NO: 3 - DNA, 22 bp, Artificial Sequence
  synthetic house-keeping gene transcript actin-beta (ACTB) RT-PCR primer
aggatgcagaaggagatcactg

SEQ ID NO: 4 - DNA, 22 bp, Artificial Sequence
  synthetic house-keeping gene transcript actin-beta (ACTB) RT-PCR primer
atactcctgcttgctgatccac

SEQ ID NO: 5 - DNA, 23 bp, Artificial Sequence
  synthetic house-keeping gene transcript ribosomal protein S9 (RPS9) RT-PCR primer
gacccttcgagaaatctcgtctc

SEQ ID NO: 6 - DNA, 22 bp, Artificial Sequence
  synthetic house-keeping gene transcript ribosomal protein S9 (RPS9) RT-PCR primer
tctcatcaagcgtcagcagttc

SEQ ID NO: 7 - DNA, 22 bp, Artificial Sequence
  synthetic interleukin 1-beta (IL1B) Q-PCR amplification primer
gtgctgaatgtggactcaatcc

SEQ ID NO: 8 - DNA, 19 bp, Artificial Sequence
  synthetic interleukin 1-beta (IL1B) Q-PCR amplification primer
accctaaggcaggcagttg

SEQ ID NO: 9 - DNA, 20 bp, Artificial Sequence
  synthetic stratifin (SFN) Q-PCR amplification primer
cctgcgaagagcgaaacctg

SEQ ID NO: 10 - DNA, 22 bp, Artificial Sequence
  synthetic stratifin (SFN) Q-PCR amplification primer
tcaatactggacagcaccctcc

SEQ ID NO: 11 - DNA, 19 bp, Artificial Sequence
  synthetic tubulin-alpha, ubiquitous (K-ALPHA-1) Q-PCR amplification primer
agcgtgcctttgttcactg

SEQ ID NO: 12 - DNA, 22 bp, Artificial Sequence
  synthetic tubulin-alpha, ubiquitous (K-ALPHA-1) Q-PCR amplification primer
cacaccaacctcctcataatcc

SEQ ID NO: 13 - DNA, 22 bp, Artificial Sequence
  synthetic dual specificity phosphatase 1 (DUSP1) Q-PCR forward (F) primer
cctaccagtattattcccgacg

SEQ ID NO: 14 - DNA, 22 bp, Artificial Sequence
  synthetic dual specificity phosphatase 1 (DUSP1) Q-PCR reverse (R) primer
ttgtgaaggcagacacctacac

SEQ ID NO: 15 - DNA, 19 bp, Artificial Sequence
  synthetic H3 histone, family 3A (H3F3A, HA3A) Q-PCR forward (F) primer
aaagcacccaggaagcaac

SEQ ID NO: 16 - DNA, 22 bp, Artificial Sequence
  synthetic H3 histone, family 3A (H3F3A, HA3A) Q-PCR reverse (R) primer
gcgaatcagaagttcagtggac

SEQ ID NO: 17 - DNA, 22 bp, Artificial Sequence
  synthetic interleukin 1, beta (IL1B) Q-PCR forward (F) primer
gtgctgaatgtggactcaatcc

SEQ ID NO: 18 - DNA, 19 bp, Artificial Sequence
  synthetic interleukin 1, beta (IL1B) Q-PCR reverse (R) primer
accctaaggcaggcagttg

SEQ ID NO: 19 - DNA, 22 bp, Artificial Sequence
  synthetic interleukin 8 (IL8, IL-8) Q-PCR forward (F) primer
gagggttgtggagaagtttttg

SEQ ID NO: 20 - DNA, 22 bp, Artificial Sequence
  synthetic interleukin 8 (IL8, IL-8) Q-PCR reverse (R) primer
ctggcatcttcactgattcttg

SEQ ID NO: 21 - DNA, 21 bp, Artificial Sequence
  synthetic ornithine decarboxylase antizyme 1 (OAZ1) Q-PCR forward (F) primer
agagagagtcttcgggagagg

SEQ ID NO: 22 - DNA, 21 bp, Artificial Sequence
  synthetic ornithine decarboxylase antizyme 1 (OAZ1) Q-PCR reverse (R) primer
agatgagcgagtctacggttc

SEQ ID NO: 23 - DNA, 22 bp, Artificial Sequence
  synthetic S100 calcium binding protein (PS(100P), S100P) Q-PCR forward (F) primer
gagttcatcgtgttcgtggctg

SEQ ID NO: 24 - DNA, 22 bp, Artificial Sequence
  synthetic S100 calcium binding protein (PS(100P), S100P) Q-PCR reverse (R) primer
ctccagggcatcatttgagtcc

SEQ ID NO: 25 - DNA, 21 bp, Artificial Sequence
  synthetic spermidine/spermine N1-acetyltransferase (SAT) Q-PCR forward (F) primer
ccagtgaagagggttggagac

SEQ ID NO: 26 - DNA, 22 bp, Artificial Sequence
  synthetic spermidine/spermine N1-acetyltransferase (SAT) Q-PCR reverse (R) primer
tggaggttgtcatctacagcag

SEQ ID NO: 27 - DNA, 20 bp, Artificial Sequence
  synthetic growth arrest and DNA-damage-inducible, beta (GADD45B) Q-PCR forward (F) primer
tgatgaatgtggacccagac

SEQ ID NO: 28 - DNA, 19 bp, Artificial Sequence
  synthetic growth arrest and DNA-damage-inducible, beta (GADD45B) Q-PCR reverse (R) primer
gagcgtgaagtggatttgc

SEQ ID NO: 29 - DNA, 22 bp, Artificial Sequence
  synthetic regulator of G-protein signaling 2, 24 kDa (RGS2) Q-PCR forward (F) primer
cctgccataaagactgaccttg

SEQ ID NO: 30 - DNA, 22 bp, Artificial Sequence
  synthetic regulator of G-protein signaling 2, 24 kDa (RGS2) Q-PCR reverse (R) primer
gcttcctgattcactacccaac

(19) United States
(12) Patent Application Publication
(10) Pub. No.: US 2009/0047667 A1
(43) Pub. Date: Feb. 19, 2009

(54) Salivary transcriptome diagnostics

(75) Inventor: David Wong, Beverly Hills, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of The University of California, Oakland, CA (US)

(21) Appl. No.: 11/632,817

(22) PCT Filed: Jul. 15, 2005
(86) PCT No.: PCT/US05/25138

§ 371 Date: May 30, 2008

Related U.S. Application Data
(60) Provisional application No. 60/589,627, filed on Jul. 21, 2004.

(51) Int. Cl. C12Q 1/68

(52) U.S. Cl. 435/6

(57) ABSTRACT

The present invention concerns probes and methods useful in diagnosing, identifying and monitoring the progression of disease states through measurements of gene products in saliva.

### GOVERNMENT INTERESTS

Pursuant to 35 U.S.C. §202(c) it is acknowledged that the U.S. Government has certain rights in the invention described herein, which was made in part with funds from the National Institutes of Health, Grant Number UO1 DE15018 and RO1 DE15018.

### FIELD OF THE INVENTION

The present invention relates generally to the detection and diagnosis of human disease states and methods relating thereto. More particularly, the present invention concerns probes and methods useful in diagnosing, identifying and monitoring the progression of disease states through measurements of gene products in saliva.

### BACKGROUND OF THE INVENTION

Saliva is not a passive “ultrafiltrate” of serum (Rehak, N. N. et al. 2000 Clin Chem Lab Med 38:335-343), but contains a distinctive composition of enzymes, hormones, antibodies, and other molecules. In the past 10 years, the use of saliva as a diagnostic fluid has been successfully applied in diagnostics and predicting populations at risk for a variety of conditions (Streckfus, C. F. & Bigler, L. R. 2002 Oral Dis 8:69-76). Diagnostic biomarkers in saliva have been identified for monitoring caries, periodontitis, oral cancer, salivary gland diseases, and systemic disorders, e.g., hepatitis and HIV (Lawrence, H. P. 2002 J Can Dent Assoc 68:170-174.).

Human genetic alterations are detectable both intracellularly and extracellularly (Sidransky, D. 1997 Science 278:1054-1058). Nucleic acids have been identified in most bodily fluids including blood, urine and cerebrospinal fluid, and have been successfully adopted for using as diagnostic biomarkers for diseases (Anker, P. et al. 1999 Cancer Metastasis Rev 18:65-73; Rieger-Christ, K. M. et al. 2003 Cancer 98:737-744; Wong, L. J. et al. 2003 Cancer Res 63:3866-3871). Recent interest has developed to detect nucleic acid markers in saliva. To date, most of the DNA or RNA in saliva was found to be of viral or bacterial origin (Stamey, F. R. et al. 2003 J Virol Methods 108:189-193; Mercer, D. K. et al. 2001 FEMS Microbiol Lett 200:163-167). There are a limited number of reports demonstrating tumor cell DNA heterogeneity in saliva of oral cancer patients (Liao P. H. et al. 2000 Oral Oncol 36:272-276; El-Naggar, A. K. et al. 2001 J Mol Diagn 3:164-170). We have not found published evidence of human mRNA detectable in saliva.

More than 1.3 million new cancer cases are expected to be diagnosed in 2004 in the United States (Cancer facts and figures 2004. Atlanta: American Cancer Society, 2004). Cancer will cause approximately 563,700 deaths of American this year, killing one person every minute. These numbers have been steadily increasing over the past ten years, despite advances in cancer treatment. Moreover, for some cancers such as oral cavity cancer, the overall 5-year survival rates have not improved in the past several decades, remaining low at approximately 30-50% (Epstein, J. B. et al. 2002 J Can Dent Assoc 68: 617-621; Mao, L. et al. 2004 Cancer Cell 5: 311-316). A critical factor in the lack of prognostic improvement is the fact that a significant proportion of cancers initially are asymptomatic lesions and are not diagnosed or treated until they reach an advanced stage. Early detection of cancer is the most effective means to reduce death from this disease.

The genetic aberrations of cancer cell lead to altered gene expression patterns, which can be identified long before the resulting cancer phenotypes are manifested. Changes that arise exclusively or preferentially in cancer, compared with normal tissue of same origin, can be used as molecular biomarkers (Sidransky, D. 2002 Nat Rev Cancer 2:210-219, 2002). Accurately identified, biomarkers may provide new avenues and constitute major targets for cancer early detection and cancer risk assessment. A variety of nucleic acid-based biomarkers have been demonstrated as novel and powerful tools for the detection of cancers (Hollstein, M. et al. 1991 Science 253:49-53; Liu, T. et al. 2000 Genes Chromosomes Cancer 27:17-25; Groden, J. et al. 1991 Cell 66:589-600). However, most of these markers have been identified either in cancer cell lines or in biopsy specimens from late invasive and metastatic cancers. We are still limited in our ability to detect cancer in its earliest stages using biomarkers. Moreover, the invasive nature of a biopsy makes it unsuitable for cancer screening in high-risk populations. This suggests an imperative need for developing new diagnostic tools that would improve early detection. The identification of molecular markers in bodily fluids that would predict the development of cancer in its earliest stage or in precancerous stage would constitute such a tool.

### SUMMARY OF THE INVENTION

The purpose of this study is to determine the transcriptome profiles in cell-free saliva obtained from normal subjects. High-density oligonucleotide microarrays were used for the global transcriptome profiling. The salivary transcriptome patterns were used to generate a reference database for salivary transcriptome diagnostics applications.

Saliva, like other bodily fluids, has been used to monitor human health and disease. This study shows that informative human mRNA exists in cell-free saliva. Salivary mRNA provides potential biomarkers to identify populations and patients at high risk for oral and systemic diseases. High-density oligonucleotide microarrays were used to profile salivary mRNA. The results demonstrated that there are thousands of human mRNAs in cell-free saliva. Quantitative PCR (Q-PCR) analysis confirmed the present of mRNA identified by our microarray study. A reference database was generated based on the mRNA profiles in normal saliva. In one embodiment of the invention, Salivary Transcriptome Diagnostics (STD) is used in disease diagnostics as well as normal health surveillance.

In another embodiment, a practical, user-friendly, room temperature protocol for the optimal preservation of salivary RNA for Salivary Transcriptome Diagnostics was developed.

### BRIEF DESCRIPTION OF THE DRAWINGS

FIG. 1. Detection of gene specific RNA in cell-free saliva using RT-PCR. (A) RNA stability in saliva tested by RT-PCR typing for actin-β (ACTB) after storage for 1, 3, and 6 months (lanes 2, 3, 4 respectively). Lane 1, molecular weight marker (100 bp ladder); Lane 5, negative control (omitting templates). (B) glyceraldehyde-3-phosphate dehydrogenase (GAPDH), ribosomal protein S9 (RPS9) and ACTB were detected consistently in all 10 cases. Lanes 1, 2 and 3 are saliva RNA, positive control (human total RNA, BD Biosciences Clontech, Palo Alto, Calif., USA) and negative controls (omitting templates), respectively.

FIG. 2. Amplification of RNA from cell-free saliva for microarray study. (A) Monitoring of RNA amplification by agarose gel electrophoresis. Lanes 1 to 5 are 1 kb DNA ladder, 5 μl saliva after RNA isolation (undetectable), 1 μl two round amplified cRNA (range from 200 bp to ˜4 kb), cRNA after fragmentation (around 100 bp) and Ambion RNA Century Marker, respectively. (B) ACTB can be detected in every main step during salivary RNA amplification. The agarose gel shows expected single band (153 bp) of PCR product. Lane 1 to 8 are 100 bp DNA ladder, total RNA isolated from cell-free saliva, 1st round cDNA, 1st round cRNA after RT, 2nd round cDNA, 2nd round cRNA after RT, positive control (human total RNA, BD Biosciences Clontech, Palo Alto, Calif., USA) and negative control (omitting templates), respectively. (C) Target cRNA analyzed by Agilent 2100 bioanalyzer before hybridization on microarray. Only one single peak in a narrow range (50-200 bp) was detected demonstrating high purity of products.

FIG. 3. Receiver operating characteristic (ROC) curve analysis for the predictive power of combined salivary mRNA biomarkers. The final logistic model included four salivary mRNA biomarkers: interleukin 1β (IL1B), ornithine decarboxylase antizyme 1 (OAZ1), spermidine/spermine N1-acetyltransferase (SAT) and interleukin 8 (IL-8). Using a cut-off probability of 50%, we obtained sensitivity of 91% and specificity of 91% by ROC. The calculated area under the ROC curve was 0.95.

FIG. 4. Classification and regression trees (CART) model assessing the salivary mRNA predictors for oral squamous cell carcinoma (OSCC). IL-8 (cutoff value=3.14E-18), chosen as the initial split, produced two child groups from the parent group containing the total 64 samples. Normal-1 group was further partitioned by SAT (cutoff value=1.13E-14), while cancer-1 group was further partitioned by H3F3A (cutoff value=2.07E-16). The 64 samples involved in this study were classified into the final cancer or normal group by CART. The overall sensitivity is 90.6% (29/32, in normal group) and specificity is 90.6% (29/32, in cancer group) for OSCC classification.

FIG. 5. Detection and quantification of human mRNA in RNAlater™-treated saliva. (A). RT-PCR was used to detect transcripts from three genes, beta-actin (ACTB), glyceraldehyde-3-phosphate dehydrogenase (GAPDH) and interleukin 8 (IL-8). (B). RNA quantification by using Ribogreen® kit (Molecular Probes) showed higher RNA yield from RNAlater™ processed sample other than the Superase-In (Ambion) processed samples.

FIG. 6. Quantitative PCR (qPCR) to quantify the salivary GAPDH and IL-8.

### DETAILED DESCRIPTION OF THE PREFERRED EMBODIMENT

The present invention concerns the early detection, diagnosis, and prognosis of human disease states. Markers of a disease state, in the form of expressed RNA molecules of specified sequences or polypeptides expressed from these RNA molecules from the saliva of individuals with the disease state, are disclosed. These markers are indicators of the disease state and, when differentially expressed relative to expression in a normal subject, are diagnostic for the presence of the disease state in patients. Such markers provide considerable advantages over the prior art in this field. Since they are detected in saliva samples, it is not necessary to suspect that an individual exhibits the disease state (such as a tumor) before a sample may be taken, and in addition, the drawing of a saliva sample is much less invasive and painful to the patient than tissue biopsy or blood drawing. The detection methods disclosed are thus suitable for widespread screening of asymptomatic individuals.

### EXAMPLE 1

### RNA Profiling of Cell-Free Saliva Using Microarray Technology

### Materials & Methods

### Normal Subjects

Saliva samples were obtained from ten normal donors from the Division of Otolaryngology, Head and Neck Surgery, at the Medical Center, University of California, Los Angeles (UCLA), Calif., in accordance with a protocol approved by the UCLA Institutional Review Board. The following inclusion criteria were used: age≧30 years; no history of malignancy, immunodeficiency, autoimmune disorders, hepatitis, HIV infection or smoking. The study population was composed of 6 males and 4 females, with an average age of 42 years (range from 32 to 55 years).

### Saliva Collection and Processing

Saliva samples were collected between 9 am and 10 am in accordance with published protocols (Navazesh, M. 1993 Ann N Y Acad Sci 694:72-77). Subjects were asked to refrain from eating, drinking, smoking or oral hygiene procedures for at least one hour prior to saliva collection. Saliva samples were centrifuged at 2,600×g for 15 min at 4° C. Saliva supernatant was separated from the cellular phase. RNase inhibitor (Superase-In, Ambion Inc., Austin, Tex., USA) and protease inhibitor (Aprotinin, Sigma, St. Louis, Mo., USA) were then added into the cell-free saliva supernatant.

RNA Isolation from Cell-Free Saliva

RNA was isolated from cell-free saliva supernatant using the modified protocol from the manufacturer (QIAamp Viral RNA kit, Qiagen, Valencia, Calif., USA). Saliva (560 μL), mixed well with AVL buffer (2,240 μL), was incubated at room temperature for 10 min. Absolute ethanol (2,240 μL) was added and the solution passed through silica columns by centrifugation at 6,000×g for 1 min. The columns were then washed twice, centrifuged at 20,000×g for 2 min, and eluted with 30 μL Rnase-free water at 9,000×g for 2 min. Aliquots of RNA were treated with RNase-free DNase (DNase I-DNA-free, Ambion Inc., Austin, Tex., USA) according to the manufacturer's instructions. The quality of isolated RNA was examined by RT-PCR for three house-keeping gene transcripts: glyceraldehyde-3-phosphate dehydrogenase (GAPDH), actin-β (ACTB) and ribosomal protein S9 (RPS9). Primers were designed using PRIMER3 software (genome.wi.mit.edu) and were synthesized commercially (Fisher Scientific, Tustin, Calif., USA) as follows: 5′ TCACCAGGGCTGCTTTTAACTC3′ (SEQ ID NO: 1) and 5′ATGACAAGCTTCCCGTTCTCAG3′ (SEQ ID NO: 2) for GAPDH; 5′AGGATGCAGAAGGAGATCACTG3′ (SEQ ID NO: 3) and 5′ATACTCCTGCTTGCTGATCCAC3′ (SEQ ID NO: 4) for ACTB; 5′GACCCTTCGAGAAATCTCGTCTC3′ (SEQ ID NO: 5) and 5′TCTCATCAAGCGTCAGCAGTTC3′ (SEQ ID NO: 6) for RPS9. The quantity of RNA was estimated using Ribogreen® RNA Quantitation Kit (Molecular Probes, Eugene, Oreg., USA).

Target cRNA Preparation

Isolated RNA was subjected to linear amplification according to published method (Ohyama, H. et al. 2000 Biotechniques 29:530-536). In brief, reverse transcription using T7-oligo-(dT)24 as the primer was performed to synthesize the first strand cDNA. The first round of in vitro transcription (IVT) was carried out using T7 RNA polymerase (Ambion Inc., Austin, Tex., USA). The BioArray™ High Yield RNA Transcript Labeling System (Enzo Life Sciences, Farmingdale, N.Y., USA) was used for the second round IVT to biotinylate the cRNA product; the labeled cRNA was purified using GeneChip® Sample Cleanup Module (Affymetrix, Santa Clara, Calif., USA). The quantity and quality of cRNA were determined by spectrophotometry and gel electrophoresis. Small aliquots from each of the isolation and amplification steps were used to assess the quality by RT-PCR. The quality of the fragmented cRNA (prepared as described by Kelly, J. J. et al. 2002 Anal Biochem 311:103-118) was assessed by capillary electrophoresis using the 2100 Bioanalyzer (Agilent Technologies, Palo Alto, Calif., USA).

### HG-U133A Microarray Analysis

The Affymetrix Human Genome U133A Array, which contains 22,215 human gene cDNA probe sets representing approximately 19,000 genes (i.e., each gene may be represented by more than one probe sets), was applied for gene expression profiling. The array data were normalized and analyzed using Microarray Suite (MAS) software (Affymetrix). A detection p-value was obtained for each probe set. Any probe sets with p<0.04 was assigned “present”, indicating the matching gene transcript is reliably detected (Affymetrix, 2001). The total number of present probe sets on each array was obtained and the present percentage (P%) of present genes was calculated. Functional classification was performed on selected genes (present on all ten arrays, p<0.01) by using the Gene Ontology Mining Tool (netaffx.com).

### Quantitative Gene Expression Analysis by Q-PCR

Q-PCR was performed using iCycler™ thermal Cycler (Bio-Rad, Hercules, Calif., USA). A 2 μL aliquot of the isolated salivary RNA (without amplification) was reverse transcribed into cDNA using MuLV Reverse Transcriptase (Applied Biosystems, Foster City, Calif., USA). The resulting cDNA (3 μL) was used for PCR amplification using iQ SYBR Green Supermix (Bio-Rad, Hercules, Calif., USA). The primers were synthesized by Sigma-Genosys (Woodlands, Tex., USA) as follows: 5′ GTGCTGAATGTGGACTCAATCC3′ (SEQ ID NO: 7) and 5′ ACCCTAAGGCAGGCAGTTG3′ (SEQ ID NO: 8) for interleukin 1-beta (IL1B); 5′ CCTGCGAAGAGCGAAACCTG 3′ (SEQ ID NO: 9) and 5′ TCAATACTGGACAGCACCCTCC 3′ (SEQ ID NO: 10) for stratifin (SFN); 5′ AGCGTGCCTTTGTTCACTG 3′ (SEQ ID NO: 11) and 5′ CACACCAACCTCCTCATAATCC 3′ (SEQ ID NO: 12) for tubulin-alpha, ubiquitous (K-ALPHA-1). All reactions were performed in triplicate with conditions customized for the specific PCR products. The initial amount of cDNA of a particular template was extrapolated from a standard curve using the LightCycler software 3.0 (Bio-Rad, Hercules, Calif., USA). The detailed procedure for quantification by standard curve has been previously described (Ginzinger, D. 2002 Exp Hematol 30:503-512).

### Results

### RNA Isolation and Amplification

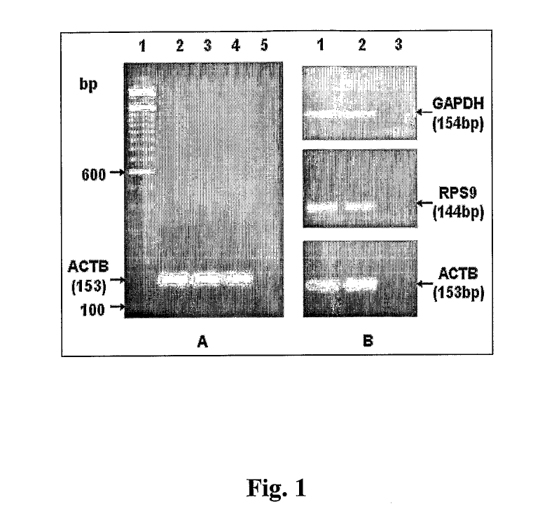

On average, 60.5±13.1 ng (n=10) of total RNA was obtained from 560 μL cell-free saliva samples (Table 1). RT-PCR results demonstrated all 10 saliva samples contain mRNAs that encode for house keeping genes: GAPDH, ACTB and RPS9. The mRNA of these genes could be preserved without significant degradation for more than 6 months at −80° C. (FIG. 1). After two rounds of T7 RNA linear amplification, the average yield of biotinylated cRNA was 42.2±3.9 μg with A260/280=2.067 (Table 1).

TABLE 1
Gene expression profiling in cell-free saliva obtained from ten normal donors

| | | | RNA | cRNA | | |
|---|---|---|---|---|---|---|
| Subject | Gender | Age | (ng)^{a} | (μg)^{b} | Present Probes^{c} | Probe P %^{d} |
| 1 | F | 53 | 60.4 | 44.3 | 3172 | 14.24 |
| 2 | M | 42 | 51.6 | 40.8 | 2591 | 11.62 |
| 3 | M | 55 | 43.2 | 34.8 | 2385 | 10.70 |
| 4 | M | 42 | 48.2 | 38.0 | 2701 | 12.12 |
| 5 | M | 46 | 60.6 | 42.7 | 3644 | 16.35 |
| 6 | M | 48 | 64.8 | 41.8 | 2972 | 13.34 |
| 7 | F | 40 | 75.0 | 44.3 | 2815 | 12.63 |
| 8 | M | 33 | 77.8 | 49.3 | 4159 | 18.66 |
| 9 | F | 32 | 48.8 | 41.4 | 2711 | 12.17 |
| 10 | F | 32 | 79.8 | 44.4 | 4282 | 19.22 |
| Mean ± SD | | 42 ± 8.3 | 60.5 ± 13.12 | 42.2 ± 3.94 | 3143 ± 665.0 | 14.11 ± 2.98 |

^{a}Total RNA quantity in 560 μL cell-free saliva supernatant
^{b}The cRNA quantity after two rounds of T7 amplification
^{c}Number of probes showing present call on HG U133A microarray (detection p < 0.04)
^{d}Present percentage (P %) = Number of probes assigned present call/Number of total probes (22,283 for HG U133A microarray)

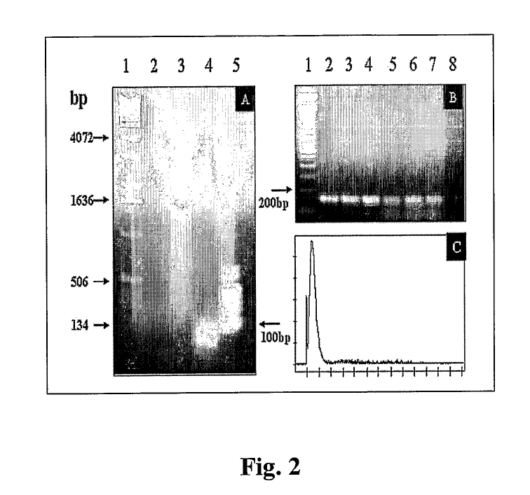

The cRNA ranged from 200 bp to 4 kb before fragmentation; and was concentrated to approximately 100 bp after fragmentation. The quality of cRNA probe was confirmed by capillary electrophoresis before the hybridizations. ACTB mRNA was detectable using PCR/RT-PCR on original sample and products from each amplification steps: first cDNA, first in vitro transcription (IVT), second cDNA and second IVT (FIG. 2).

Microarray Profiling of Salivary mRNA

Salivary mRNA profiles of ten normal subjects were obtained using HG U133A array which contains 22,283 cDNA probes. An average of 3,143±665.0 probe sets (p<0.04) were found on each array (n=10) with assigned present calls. These probe sets represent approximately 3,000 different mRNAs. The average present call percentage was 14.11±2.98% (n=10). A reference database which includes data from the ten arrays was generated. The probe sets representing GAPDH, ACTB and RPS9 assigned present calls on all 10 arrays. There were totally 207 probe sets representing 185 genes assigned present calls on all 10 arrays with detection p<0.01. These genes were categorized on the basis of their known roles in biological processes and molecular functions (Table 2). The major functions of the 185 genes are related to cell growth/maintenance (119 genes), molecular binding (118 genes) and cellular structure composition (95 genes). These were termed as “Normal Salivary Core Transcriptome (NSCT)”.

TABLE 2
Biological processes and molecular functions of 185
genes in cell-free saliva from ten normal donors (data
obtained by using Gene Ontology Mining Tool)

| | Genes, | | Gene, |
|---|---|---|---|
| Biological process^{a} | n^{b} | Molecular function^{a} | n^{b} |
| Cell growth and/or | 119 | Binding | 118 |
| maintenance | | Nucleic acid binding | 89 |
| Metabolism | 93 | RNA binding | 73 |
| Biosynthesis | 70 | Calcium ion binding | 12 |
| Protein metabolism | 76 | Other binding | 23 |
| Nucleotide metabolism | 10 | Structural molecule | 95 |
| Other metabolisms | 18 | Ribosomal constituent | 73 |
| Cell organization and | 2 | Cytoskeleton constituent | 17 |
| biogenesis | | Muscle constituent | 2 |
| Homeostasis | 3 | Obsolete | 15 |
| Cell cycle | 5 | Transporter | 4 |
| Cell proliferation | 11 | Enzyme | 20 |
| Transport | 5 | Signal transduction | 10 |
| Cell motility | 8 | Transcription regulator | 7 |
| Cell communication | 34 | Translation regulator | 5 |
| Response to external | 19 | Enzyme regulator | 9 |
| stimulus | | Cell adhesion molecule | 1 |
| Cell adhesion | 3 | Molecular function unknown | 6 |
| Cell-cell signaling | 5 | | |
| Signal transduction | 17 | | |
| Obsolete | 8 | | |
| Development | 18 | | |
| Death | 2 | | |
| Biological process | 11 | | |
| unknown | | | |

^{a}One gene may have multiple molecular functions or participate in different biological processes.
^{b}Number of genes classified into a certain group/subgroup.

### Q-PCR Validation and Quantitation Analysis

Real time quantitative PCR (Q-PCR) was used to validate the presence of human mRNA in saliva by quantifying selected genes from the 185 “Normal Salivary Core Transcriptome” genes. IL1B, SFN and K-ALPHA-1 were randomly selected and assigned present calls on all 10 arrays, for validation. Q-PCR results showed that mRNA of IL1B, SFN and K-ALPHA-1 were detectable in all 10 original, unamplified, cell-free saliva. The relative amounts (in copy number) of these transcripts (n=10) were: 8.68×10^{3±4.15×10}³ for IL1B; 1.29×10^{5±1.08×10}⁵ for SFN; and 4.71×10⁶±8.37×10⁵ for K-ALPHA-1. The relative RNA expression levels of these genes measured by Q-PCR were similar to those measured by the microarrays.

Saliva meets the demands of an inexpensive, non-invasive and accessible bodily fluid to act as an ideal diagnostic medium. Specific and informative biomarkers in saliva are greatly needed to serve for diagnosing disease and monitoring human health (Bonassi, S. et al. 2001 Mutat Res 480-481:349-358; Streckfus, C. F. et al. 2002 Oral Dis 8:69-76; Sidransky, D. 2000 Nat Reviews 3:210-219). Knowing the constituents in saliva is essential for using this medium to identify potential biomarkers for disease diagnostics (Pusch, W. et al. 2003 Pharmacogenomics 4:463-476). Prior to this invention, one criticism was the idea that informative molecules are generally present in low amounts in saliva. However, with new amplification techniques and highly sensitive assays, this may no longer be a limitation (Xiang, C. C. et al 2003 Nucleic Acids Res 31:e53). In the present Example, the human RNA was successfully isolated from cell-free saliva supernatant. The quality of salivary mRNA was proved to be sufficient for use in RT-PCR, Q-PCR and microarray experiments.

Distinct difference exists between saliva and other bodily fluids (e.g., blood) in that saliva naturally contains microorganisms (Sakki, T. & Knuuttila, M. 1996 Eur J Oral Sci 104:619-622). In addition, some extraneous substances (e.g., food debris) make the composition of saliva more complex. Therefore, it is simpler and more accurate to use the fluid/supernatant phase of saliva, instead of the whole saliva as medium for detecting biomarkers. In this Example, the conditions for separating the pellet and saliva supernatant were optimized to avoid mechanical rupture of cellular elements which would contribute to the RNA detected in the fluidic cell-free phase (St. John, M. A. R. et al. 2004, in press). These results demonstrate that it is feasible and efficient to use cell-free saliva for transcriptome analysis. While it is a novel finding that human mRNAs exist in cell-free saliva supernatant, nucleic acids have long been detected in other cell-free bodily fluids and subsequently used for disease diagnostics (Sidransky, D. 1997 Science 278:1054-1058). For example, specific oncogene, tumor suppressor gene and microsatellite alterations have been identified in patients' serum (Anker, P. et al. 2003 Int J Cancer 103:149-152). Moreover, tumor mRNAs have been isolated and amplified from serum of patients with different malignancies (Kopreski, M. S. et al. 1999 Clin Cancer Res 5:1961-1965; Fleischhacker, M. et al 2001 Ann NY Acad Sci 945:179-188). It has been widely accepted that these genomic messengers detected extracellularly can serve as biomarkers for diseases (Sidransky, D. 1997 Science 278:1054-1058).

To our knowledge, this is the first report where human mRNA in saliva is globally profiled. Using microarray technology, we discovered that approximately 3,000 different human mRNAs exist in cell-free saliva of each normal subject. The salivary transcriptome pattern in cell-free saliva from normal populations is envisioned to serve as a health-monitoring database. It should be noted that we now know the human genome composed of more than 30,000 genes (Venter J. C. et al. 2001 Science 291:1304-1351) and the probe sets on HG U133A microarray used in this Example represent only ˜19,000 human genes, additional gene transcripts not detectable by the HG U133A microarray, are predicted to exist in the cell-free saliva and can be detected using our invention. The identified gene transcripts in this Example, particularly the Normal Salivary Core Transcriptome (NSCT) mRNAs, represent the common transcriptome of normal cell-free saliva. We envision that different, informative and diagnostic transcriptome can be identified in saliva from patients with various disease conditions. Therefore, human salivary mRNA is envisioned to be used as diagnostic biomarkers for oral and systemic diseases that are manifested in the oral cavity.

In one embodiment of the invention the salivary transcriptome diagnostics is used to monitor health of normal patients. In another embodiment, the salivary transcriptome diagnostics is used to detect markers for diseases for early diagnosis for cancers (e.g., prostate, colon, breast, lung, oral, etc.), as well as for systemic diseases, such as autoimmune diseases, diabetes, osteoporosis; neurological diseases, such as Alzheimer's disease, Parkinson's disease, etc.

### EXAMPLE 2

### Salivary Transcriptome Diagnostics for Oral Cancer Detection

Purpose: Oral fluid (saliva) meets the demand for non-invasive, accessible and highly-efficient diagnostic medium. Our discovery that a large panel of human RNA can be reliably detected in saliva gives rise to a novel clinical approach, Salivary Transcriptome Diagnostics. In this Example we evaluate the diagnostic value of this new approach by using oral squamous cell carcinoma (OSCC) as the proof-of-principle disease.

It has been shown that identical mutation present in the primary tumor can be identified in the bodily fluids tested from affected patients (Sidransky, D. 1997 Science 278:1054-1059). Cancer related nucleic acids in blood, urine and cerebrospinal fluid (CSF) has been used as biomarkers for cancer diagnosis (Anker, P. et al 1999 Cancer Metastasis Rev 18:65-73; Rieger-Christ, K. M. et al. 2003 Cancer 98:737-744; Wong, L. J. et al. 2003 Cancer Res 63:3866-3871). More recently, mRNA biomarkers in serum or plasma have been targets for RT-PCR-based detection strategies in patients with cancers (Kopreski, M. S. et al. 2001 Ann N Y Acad Sci 945:172-178; Bunn, P. J., Jr. 2003 J Clin Oncol 21:3891-3893). Parallel to the increasing number of such biomarkers in bodily fluids is the growing availability of technologies using more powerful and cost-efficient methods that enable mass screening for genetic alterations. Our discovery by microarray technology that a large panel of human mRNA exists in saliva (Example 1) provides a novel clinical approach, Salivary Transcriptome Diagnostics, for applications in disease diagnostics as well as for normal health surveillance. It is a high throughput, robust and reproducible approach to harness RNA signatures from saliva. Moreover, using saliva as a diagnostic fluid meets the demands for inexpensive, non-invasive and accessible diagnostic methodology (Lawrence, H. P. 2002 J Can Dent Assoc 68:170-174, 2002). In this Example, we tested the hypothesis that distinct mRNA expression patterns can be identified in saliva from cancer patients, and the differentially expressed transcripts can serve as biomarkers for cancer detection. The proof-of-principle disease in this study is oral squamous cell carcinoma (OSCC). The rationale is that oral cancer cells are immersed in the salivary milieu and genetic heterogeneity has been detected in saliva from patients with OSCC (El-Naggar, A. K et al. 2001 J Mol Diagn 3:164-170; Liao, P. H et al. 2000 Oral Oncol 36:272-276, 2000).

Experimental Design: Unstimulated saliva was collected from patients (n=32) with primary T1/T2 OSCC and normal subjects (n=32) with matched age, gender and smoking history. RNA isolation was performed from the saliva supernatant, followed by two-round linear amplification using T7 RNA polymerase. Human Genome U133A microarrays were applied for profiling human salivary transcriptome. The different gene expression patterns were analyzed by combining a t test comparison and a fold-change analysis on ten matched cancer patients and controls. Quantitative PCR (qPCR) was used to validate the selected genes that showed significant difference (P<0.01) by microarray. The predicting power of these salivary mRNA biomarkers were analyzed by receiver operating characteristic curve and classification models.

Results: Microarray analysis showed 1,679 genes which exhibited significantly different expression level in saliva between cancer patients and controls (P<0.05). Seven cancer-related RNA biomarkers, that exhibited at least 3.5-fold elevation in OSCC saliva (P<0.01), were consistently validated by qPCR on saliva samples from OSCC patients (n=32) and controls (n=32). These salivary RNA biomarkers are transcripts of interleukin 8 (IL-8), interleukin 1-beta (IL1B), dual specificity phosphatase 1 (DUSP1), H3 histone, family 3A (HA3A), ornithine decarboxylase antizyme 1 (OAZ1), S100 calcium binding protein PS (100P) and spermidine/spermine N1-acetyltransferase (SAT). The combinations of these biomarkers yielded sensitivity (91%) and specificity (91%) in distinguishing OSCC from the controls.

Conclusions: The utility of salivary transcriptome diagnostics was successfully demonstrated in this study for oral cancer detection. This novel clinical approach is envisioned as a robust, high-throughput and reproducible tool for early cancer detection. Salivary transcriptome profiling is envisioned to be applied to evaluate other major diseases as well as normal health surveillance.

### Patients and Methods

Patient Selection. Oral squamous cell carcinoma (OSCC) patients were recruited from Medical Centers at University of California, Los Angeles (UCLA); University of Southern California (USC), Los Angeles, Calif.; and University of California San Francisco (UCSF), San Francisco, Calif. Thirty-two patients with documented primary T1 or T2 OSCC were included in this study. All patients had recently been diagnosed with primary disease, and had not received any prior treatment in the form of chemotherapy, radiotherapy, surgery, or alternative remedies. An equal number of age and sex matched subjects with comparable smoking histories were selected as a control group (St. John, M. A. R et al. 2004 IL-6 and IL-8: Potential Biomarkers for Oral Cavity and Oropharyngeal SCCA. Archives of Otolaryngology-Head & Neck Surgery, in press). Among the two subject groups, there were no significant differences in terms of mean age: OSCC patients, 49.8±7.6 years; normal subjects, 49.1±5.9 years (Student's t test P>0.80); gender (P>0.90); or smoking history (P>0.75). No subjects had a history of prior malignancy, immunodeficiency, autoimmune disorders, hepatitis, or HIV infection. All subjects signed the Institutional Review Board approved consent form agreeing to serve as saliva donors for the experiments.

Saliva collection and RNA isolation. Unstimulated saliva samples were collected between 9 am and 10 am with previously established protocols (Navazesh, M. 1993 Ann N Y Acad Sci 694:72-77). Subjects were asked to refrain from eating, drinking, smoking or oral hygiene procedures for at least one hour prior to the collection. Saliva samples were centrifuged at 2,600×g for 15 min at 4° C. The supernatant was removed from the pellet and treated with RNase inhibitor (Superase-In, Ambion Inc., Austin, Tex.). RNA was isolated from 560 μl of saliva supernatant using QIAamp Viral RNA kit (Qiagen, Valencia, Calif.). Aliquots of isolated RNA were treated with RNase-free DNase (DNaseI-DNA-free, Ambion Inc., Austin, Tex.) according to the manufacturer's instructions. The quality of isolated RNA was examined by RT-PCR for three cellular maintenance gene transcripts: glyceraldehyde-3-phosphate dehydrogenase (GAPDH), actin-β (ACTB) and ribosomal protein S9 (RPS9). Only those samples exhibiting PCR products for all three genes were used for subsequent analysis.

Microarray analysis. Saliva from ten OSCC patients (7 male, 3 female, age=52±9.0) and from ten gender and age matched normal donors (age=49±5.6) was used for microarray study. Isolated RNA from saliva was subjected to linear amplification by RiboAmp™ RNA Amplification kit (Arcturus, Mountain View, Calif.). The RNA amplification efficiency was measured by using control RNA of known quantity (0.1 μg) running in parallel with the 20 samples in five independent runs. Following protocols described in Example 1, the Affymetrix Human Genome U133A Array (HG U133A, Affymetrix, Santa, Clara, Calif.) was applied for gene expression analysis.

The arrays were scanned and the fluorescence intensity was measured by Microarray Suit 5.0 software (Affimetrix, Santa Clara, Calif.) and then were imported into DNA-Chip Analyzer software for normalization and model-based analysis (Li, C. & Wong, W. H. 2001 PNAS USA 98:31-36). S-plus 6.0 (Insightful, Seattle, Wash.) was used to carry out all statistical tests. Three criteria were used to determine differentially expressed transcripts. First, we excluded probe sets on the array that were assigned as “absent” call in all samples. Second, a two-tailed student's t test was used for comparison of average gene expression signal intensity among the OSCCs (n=10) and controls (n=10). The critical alpha level of 0.05 was defined for statistical significance. Third, fold ratios were calculated for those gene transcripts that showed statistically significant difference (P<0.05). Only those gene transcripts that exhibited at least 2-fold change will be included for further analysis.

Quantitative PCR validation. qPCR was performed to validate a subset of differently expressed transcripts identified by microarray analysis. Using MuLV reverse transcriptase (Applied Biosystems, Foster City, Calif.) and random hexamers as primer (ABI, Foster City, Calif.), we synthesized cDNAs from the original and un-amplified salivary RNA. The qPCR reactions were performed in an iCycler™ PCR system (Bio-Rad, Hercules, Calif., USA), iQ SYBR Green Supermix (Bio-Rad, Hercules, Calif.). Primer sets were designed by using PRIMER3 software. All of the reactions were performed in triplicate with customized conditions for specific products. The initial amount of cDNA/RNA of a particular template was extrapolated from the standard curve as described previously (Ginzinger, D. G. 2002 Exp Hematol 30:503-512). This validation completed by testing all of the samples (n=64) including those 20 previously used for microarray study. Wilcoxon Signed Rank test was used for statistical analysis.

Receiver operating characteristic (ROC) curve analysis and prediction models. Utilizing the RT-qPCR results, ROC curve analyses (Grunkemeier, G. L. & Jin, R. 2001 Ann Thorac Surg 72:323-326) were conducted by S-plus 6.0 to evaluate the predictive power of each of the biomarkers. The optimal cutpoint was determined for each biomarker by searching for those that yielded the maximum corresponding sensitivity and specificity. ROC curves were then plotted on the basis of the set of optimal sensitivity and specificity values. Area under the curve was computed via numerical integration of the ROC curves. The biomarker that has the largest area under the ROC curve was identified as having the strongest predictive power for detecting OSCC.

Next, multivariate classification models were constructed to determine the best combination of salivary markers for cancer prediction. Firstly, using the binary outcome of the disease (OSCC) and non-disease (normal) as dependent variables, a logistic regression model was constructed controlling for patient age, gender, and smoking history. The backward stepwise regression (Renger, R. & Meadows, L. M. 1994 Acad Med 69:738) was used to find the best final model. Leave-one out cross validation was used to validate the logistic regression model. The cross validation strategy first removes one observation and then fits a logistic regression model from the remaining cases using all markers. Stepwise model selection was used for each of these models to remove variables that do not improve the model. Subsequently, the marker values were used for the case that was left out to compute a predicted class for that observation. The cross validation error rate was then the number of samples predicted incorrectly divided by the number of samples. The ROC curve was then computed for the logistic model by a similar procedure, using the fitted probabilities from the model as possible cut-points for computation of sensitivity and specificity.

Secondly, a tree-based classification model, classification and regression trees (CART), was constructed by S-plus 6.0 using the validated mRNA biomarkers as predictors. CART fits the classification model by binary recursive partitioning, in which each step involves searching for the predictor variable that results in the best split of the cancer versus the normal groups (Lemon, S. C. et al. 2003 Ann Behav Med 26:172-181). CART used the entropy function with splitting criteria determined by default settings for S-plus. By this approach, the parent group containing the entire samples (n=64) was subsequently divided into cancer groups and normal groups. The initial tree was pruned to remove all splits that did not result in sub-branches with different classifications.

### Results

On average, 54.2±20.1 ng (n=64) of total RNA was obtained from 560 μl saliva supernatant. There was no significant difference in total RNA quantity between the OSCC and the age and gender matched controls (t test, P=0.29, n=64). RT-PCR results demonstrated that all of the saliva samples (n=64) contain transcripts from three genes (GAPDH, ACTB and RPS9), which were used as quality controls for human salivary RNAs (see Example 1). A consistent amplifying magnitude (658±47.2, n=5) could be obtained after two rounds of RNA amplification. On average, the yield of biotinylated cRNA was 39.3±6.0 μg (n=20). There were no significant differences of the cRNA quantity yielded between the OSCC and the controls (t test, P=0.31, n=20).

The HG U133A microarrays were used to identify the difference in salivary profiles RNA between cancer patients and matched normal subjects. Among the 10,316 transcripts included by the previously described criteria, 1,679 transcripts with P value less than 0.05 were identified. Among these transcripts, 836 were up-regulated and 843 were down-regulated in the OSCC group. These transcripts observed were unlikely to be attributable to chance alone (x² test, P<0.0001) considering the false positives using P<0.05. Using a predefined criteria of a change in regulation>3-fold in all 10 OSCC saliva specimens, and a more stringent cutoff of P value<0.01, we identified 17 transcripts as presented in Table 3. These 17 salivary mRNAs were all up-regulated in OSCC saliva, whereas there were no mRNAs found down-regulated using the same filtering criteria. The biological functions of these genes are presented in Table 3.

TABLE 3
Salivary mRNA up-regulated (>3-fold, P < 0.01)
in OSCC identified by microarray.

| Gene | | GenBank | | |
|---|---|---|---|---|
| Symbol | Gene Name | Acc. No. | Locus | Gene functions |
| B2M | Beta-2-microglobulin | NM_004048 | 15q21- | anti-apoptosis, antigen |
| | | | q22.2 | presentation |
| DUSP1 | Dual specificity | NM_004417 | 5q34 | protein modification, |
| | phosphatase 1 | | | signal transduction, |
| | | | | oxidative stress |
| FTH1 | Ferritin, heavy | NM_002032 | 11q13 | iron ion transport, cell |
| | polypeptide 1 | | | proliferation |
| G0S2 | Putative lymphocyte | NM_015714 | 1q32.2- | cell growth and/or |
| | G0/G1 switch gene | | q41 | maintenance, |
| | | | | regulation of cell |
| | | | | cycle |
| GADD45B | Growth arrest and | NM_015675 | 19p13.3 | kinase cascade, |
| | DNA-damage- | | | apoptosis |
| | inducible, beta | | | |
| H3F3A | H3 histone, family 3A | BE869922 | 1q41 | DNA binding activity |
| HSPC016 | Hypothetical protein | BG167522 | 3p21.31 | unknown |
| | HSPC016 | | | |
| IER3 | Immediate early | NM_003897 | 6p21.3 | embryogenesis, |
| | response 3 | | | morphogenesis, |
| | | | | apoptosis, cell growth |
| | | | | and maintenance |
| IL1B | Interleukin 1, beta | M15330 | 2q14 | signal transduction, |
| | | | | proliferation, |
| | | | | inflammation, |
| | | | | apoptosis |
| IL8 | Interleukin 8 | NM_000584 | 4q13-q21 | angiogenesis, |
| | | | | replication, calcium- |
| | | | | mediated signaling |
| | | | | pathway, cell |
| | | | | adhesion, chemotaxis, |
| | | | | cell cycle arrest, |
| | | | | immune response |
| MAP2K3 | Mitogen-activated | AA780381 | 17q11.2 | signal transduction, |
| | protein kinase kinase 3 | | | protein modification |
| OAZ1 | Ornithine | D87914 | 19p13.3 | polyamine |
| | decarboxylase | | | biosynthesis |
| | antizyme 1 | | | |
| PRG1 | Proteoglycan 1, | NM_002727 | 10q22.1 | proteoglycan |
| | secretory granule | | | |
| RGS2 | Regulator of G-protein | NM_002923 | 1q31 | oncogenesis, g-protein |
| | signaling 2, 24 kDa | | | signal transduction |
| S100P | S100 calcium binding | NM_005980 | 4p16 | protein binding, |
| | protein P | | | calcium ion binding |
| SAT | Spermidine/spermine | NM_002970 | Xp22.1 | enzyme, transferase |
| | N1-acetyltransferase | | | activity |
| | EST, Highly similar | BG537190 | | iron ion homeostasis, |
| | Ferritin light chain | | | ferritin complex |

The human Genome U133A microarrays were used to identify the difference in RNA expression patterns in saliva from ten cancer patients and ten matched normal subjects. Using a criteria of a change in regulation >3-fold in all OSCC saliva specimens, and a cutoff of P value <0.01, 17 mRNA were identified, showing significant up-regulation in OSCC saliva

Quantitative PCR was performed to validate the microarray findings on an enlarged sample size including saliva from 32 patients with OSCC and 32 matched controls. Nine candidates of salivary mRNA biomarkers: DUSP1, GADD45B, H3F3A, IL1B, IL8, OAZ1, RGS2, S100P and SAT were selected based on their reported cancer association (Table 3). Table 4 presents their quantitative alterations in saliva from OSCC patients determined by qPCR. The results confirmed that transcripts of 7 of the 9 candidate mRNA (78%), DUSP1, H3F3A, IL1B, IL8, OAZ1, S100P and SAT, were significantly elevated in the saliva of OSCC patient (Wilcoxon Signed Rank test, P<0.05). The statistically significant differences in the amount of RGS2 (P=0.149) and GADD45B (P=0.116) by qPCR was not detected. The validated seven genes could be classified in three ranks by the magnitude of increase: high up-regulated mRNA including IL8 (24.3-fold); moderate up-regulated mRNA including H3F3A (5.61-fold), IL1B (5.48) and S100P (4.88-fold); and low up-regulated mRNA including DUSP1 (2.60-fold), OAZ1 (2.82-fold) and SAT (2.98-fold). The detailed statistics of the area under the receiver operator characteristics (ROC) curves, the threshold values, and the corresponding sensitivities and specificities for each of the seven potential salivary mRNA biomarkers for OSCC are listed in Table 5. The data showed IL-8 mRNA performed the best among the seven potential biomarkers for predicting the presence of OSCC. The calculated area under the ROC curve for IL-8 was 0.85. With a threshold value of 3.19E-18 mol/L, IL-8 mRNA in saliva yields a sensitivity of 88% and a specificity of 81% to distinguish OSCC from the normal.

TABLE 4

Quantitative PCR validation of selected 9 transcripts in saliva
(n = 64)^{a}

| | | | | |
|---|---|---|---|---|
| | | | | Mean |
| Gene | | | P | fold |
| symbol | Primer sequence (5′to 3′) | Validated | value | increase |

| | | | | | |
|---|---|---|---|---|---|
| DUSP1 | F: CCTACCAGTATTATTCCCGACG | (SEQ ID NO: 13) | Yes | 0.039 | 2.60 |
| | R: TTGTGAAGGCAGACACCTACAC | (SEQ ID NO: 14) | | | |
| H3F3A | F: AAAGCACCCAGGAAGCAAC | (SEQ ID NO: 15) | Yes | 0.011 | 5.61 |
| | R: GCGAATCAGAAGTTCAGTGGAC | (SEQ ID NO: 16) | | | |
| IL1B | F: GTGCTGAATGTGGACTCAATCC | (SEQ ID NO: 17) | Yes | 0.005 | 5.48 |
| | R: ACCCTAAGGCAGGCAGTTG | (SEQ ID NO: 18) | | | |
| IL8 | F: GAGGGTTGTGGAGAAGTTTTTG | (SEQ ID NO: 19) | Yes | 0.000 | 24.3 |
| | R: CTGGCATCTTCACTGATTCTTG | (SEQ ID NO: 20) | | | |
| OAZ1 | F: AGAGAGAGTCTTCGGGAGAGG | (SEQ ID NO: 21) | Yes | 0.009 | 2.82 |
| | R: AGATGAGCGAGTCTACGGTTC | (SEQ ID NO: 22) | | | |
| S100P | F: GAGTTCATCGTGTTCGTGGCTG | (SEQ ID NO: 23) | Yes | 0.003 | 4.88 |
| | R: CTCCAGGGCATCATTTGAGTCC | (SEQ ID NO: 24) | | | |
| SAT | F: CCAGTGAAGAGGGTTGGAGAC | (SEQ ID NO:25) | Yes | 0.005 | 2.98 |
| | R: TGGAGGTTGTCATCTACAGCAG | (SEQ ID NO: 26) | | | |
| GADD45B | F: TGATGAATGTGGACCCAGAC | (SEQ ID NO: 27) | No | 0.116 | |
| | R: GAGCGTGAAGTGGATTTGC | (SEQ ID NO: 28) | | | |
| RGS2 | F: CCTGCCATAAAGACTGACCTTG | (SEQ ID NO: 29) | No | 0.149 | |
| | R: GCTTCCTGATTCACTACCCAAC | (SEQ ID NO: 30) | | | |

qPCR were performed to validate the microarray findings on an enlarged sample size including saliva from 32 patients with OSCC and 32 matched control subjects. Nine potential salivary mRNA biomarkers were selected from the 17 candidates shown in Table 3. Seven of them were validated by qPCR (P <0.05). Sample includes 32 saliva from OSCC patients and 32 from matched normal subjects.
Wilcoxon's Signed Rank test: if P <0.05, validated (Yes); if P ≧0.05 not validated (No).

TABLE 5
Receiver operator characteristic (ROC) curve analysis
of OSCC associated salivary mRNA biomarkers

| | | Threshold/ | Sensi- | Speci- | |
|---|---|---|---|---|---|
| | Area under | Cutoff | tivity | ficity | Selected |
| Biomarker | ROC Curve | (M) | (%) | (%) | References |
| DUSP1 | 0.65 | 8.35E−17 | 59 | 75 | (34) |
| H3F3A | 0.68 | 1.58E−15 | 53 | 81 | (54) |
| IL1B | 0.70 | 4.34E−16 | 63 | 72 | (44) |
| IL8 | 0.85 | 3.19E−18 | 88 | 81 | (55) |
| OAZ1 | 0.69 | 7.42E−17 | 100 | 38 | (37) |
| S100P | 0.71 | 2.11E−15 | 72 | 63 | (40) |
| SAT | 0.70 | 1.56E−15 | 81 | 56 | (35) |

Utilizing the qPCR results, we conducted ROC curve analyses to evaluate the predictive power of each of the biomarkers. The optimal cutpoint was determined yielding the maximum corresponding sensitivity and specificity. The biomarker that has the largest area under the ROC curve was identified as having the strongest predictive power for detecting OSCC.

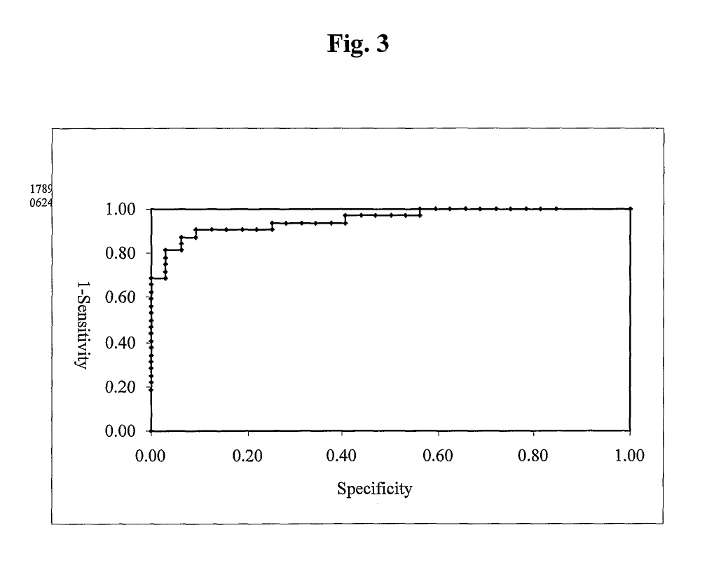

To demonstrate the utility of salivary mRNAs for disease discrimination, two classification/prediction models were examined. A logistic regression model was built based on the four of the seven validated biomarkers, IL1B, OAZ1, SAT and IL-8, which in combination provided the best prediction (Table 6). The coefficient values were positive for these four markers, indicating that the synchronized rise in their concentrations in saliva increased the probability that the sample was obtained from an OSCC subject. The leave-one-out cross-validation error rate based on logistic regression models was 19% (12/64). All but one (out of the 64) of the models generated in the leave-one-out analysis used the same set of four markers found to be significant in the full data model specified in Table 6. The ROC curve was computed for the logistic regression model. Using a cutoff probability of 50%, a sensitivity of 91% and a specificity of 91% were obtained. The calculated area under the ROC curve was 0.95 for the logistic regression model (FIG. 3).

TABLE 6
Salivary mRNA biomarkers for OSCC selected
by logistic regression model

| Biomarker | Coefficient Value | Standard Error | P value |
|---|---|---|---|
| Intercept | −4.79 | 1.51 | 0.001 |
| IL1B | 5.10E+19 | 2.68E+19 | 0.062 |
| OAZ1 | 2.18E+20 | 1.08E+20 | 0.048 |
| SAT | 2.63E+19 | 1.10E+19 | 0.020 |
| IL-8 | 1.36E+17 | 4.75E+16 | 0.006 |

The logistic regression model was built based on the four of seven validated biomarkers (IL1B, OAZ1, SAT and IL-8) that, in combination, provided the best prediction. The coefficient values are positive for these four markers, indicating that the synchronized increase in their concentration in saliva increases the probability that the sample was obtained from an OSCC subject.

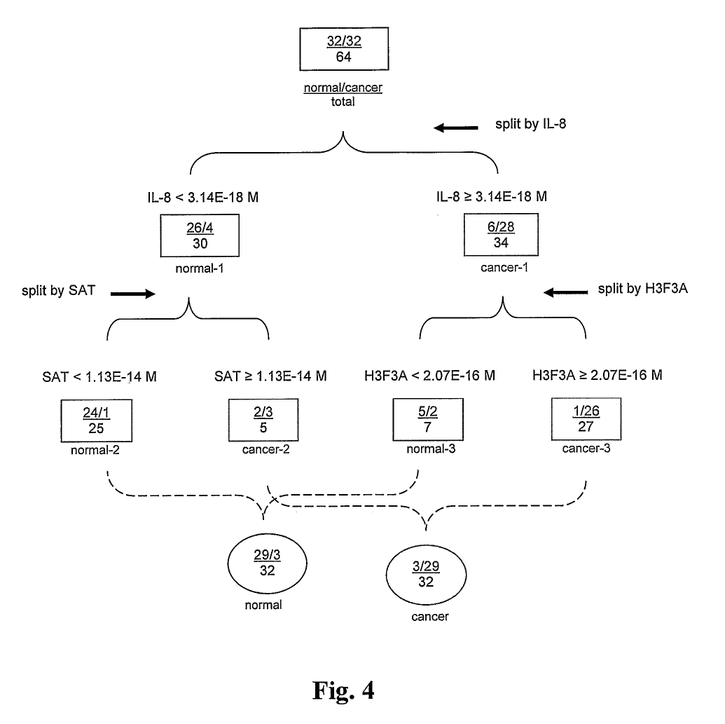

A second model, the “classification and regression trees (CART) model”, was generated (FIG. 4). The fitted CART model used the salivary mRNA concentrations of IL-8, H3F3A and SAT as predictor variables for OSCC. IL-8, chosen as the initial split, with a threshold of 3.14E-18 mol/L, produced two child groups from the parent group containing the total 64 samples. 30 samples with the IL-8 concentration<3.14E-18 mol/L were assigned into “Normal-1” group, whereas 34 with IL-8 concentration>3.14E-18 mol/L were assigned into “Cancer-1”. The “Normal-1” group was further partitioned by SAT with a threshold of 1.13E-14 mol/L. The resulting subgroups: “Normal-2”, contained 25 samples with SAT concentration≦1.13E-14 mol/L; and “Cancer-2”, contained 5 samples with SAT concentration≧1.13E-14 mol/L. Similarly, the “Cancer-1” group was further partitioned by H3F3A with a threshold of 2.07E-16 mol/L. The resulting subgroups: “Cancer-3”, contained 27 samples with H3F3A concentration≧2.07E-16 mol/L; and “Normal-3” group, contained 7 samples with H3F3A concentration<2.07E-16 mol/L. Consequently, the 64 saliva samples involved in this study were classified into the “Cancer” group and the “Normal” group by CART analysis. The “Normal” group was composed of the samples from “Normal-2” group and those from “Normal-3” group. There were a total of 32 samples assigned in the “Normal” group, 29 from normal subjects and 3 from cancer patients. Thus, by using the combination of IL-8, SAT, and H3F3A for OSCC prediction, the overall sensitivity is 90.6% (29/32). The “Cancer” group was composed of the samples from “Cancer-2” group and “Cancer-3” group. There were a total of 32 samples assigned in the final “Cancer” group, 29 from cancer patients and 3 from normal subjects. Therefore, by using the combination of these three salivary mRNA biomarkers for OSCC prediction, the overall specificity is 90.6% (29/32).

The goal of a cancer-screening program is to detect tumors at a stage early enough that treatment is likely to be successful. Screening tools are needed that exhibit the combined features of high sensitivity and high specificity. Moreover, the screening tool must be sufficiently noninvasive and inexpensive to allow widespread applicability. Significant development of biotechnology and improvement in our basic understanding of the cancer initiation and progression now enable to identify tumor signatures, such as oncogenes and tumor-suppressor gene alterations, in bodily fluids that drain from the organs affected by the tumor (Sidransky, D. 1997 Science 278:1054-1059). The results presented in this Example show that salivary transcriptome diagnostics is a suitable tool for the development of noninvasive diagnostic, prognostic and follow-up tests for cancer.

Previous studies have shown that human DNA biomarkers can be identified in saliva and used for oral cancer detection (El-Naggar, A. K et al. 2001 J Mol Diagn 3:164-170; Liao, P. H. et al. 2000 Oral Oncol 36:272-276). The presence of human mRNA in saliva expands the repertoire of diagnostic analytes for translational and clinical applications. However, RNA is more labile than DNA and is presumed to be highly susceptible to degradation by RNases. Furthermore, RNase activity in saliva is reported to be elevated in patients with cancer (Kharchenko, S. V. & Shpakov, A. A. 1989 Izv Akad Nauk SSSR Biol 58-63). It has thus been commonly presumed that human mRNA could not survive extracellularly in saliva. Surprisingly, using RT-PCR, the inventors consistently detected human mRNA in saliva, thus opening the door to saliva-based expression profiling. Using the described collection and processing protocols, the presence of control RNAs was confirmed in all saliva (patients and controls) by RT-PCR/qPCR. The quality of RNA could meet the demand for PCR, qPCR and microarray assays. In this Example, we employed prompt addition of RNase inhibitors to freshly collected oral fluids followed by ultra low temperature storage (−80° C.).

Our reported findings will bring substantial interests to the field of cancer and disease diagnostics. The interests stem not only from the fact that a saliva-based diagnostic and screening test for cancer is a simple and attractive concept, but also from the fact that conventional diagnostic cancer tests tend to be imperfect. Using oral cancer as an example, the clearly disappointing survival rate may most probably attribute to diagnostic delay (Wildt, J. et al. 1995 Clin Otolaryngol 20:21-25). Since most oral cancers arise as asymptomatic small lesions at their early stage, only when the clinician or patient notes abnormal tissues do formal diagnosis procedures begin (Epstein, J. B. et al. 2002 J Can Dent Assoc 68:617-621). Microscopic level for the progressive cancer is often too late for successful intervention (Fong, K. M. et al. 1999 in: In: S. S. HD and G. AF (eds.), Molecular Pathology of Early Cancer, pp. 13-26: IOS Press). It is also impractical to use imaging techniques for cancer screening, since they are time-consuming and expensive. These techniques are typically used for confirmation because of their insensitivity for small lesions (Myers, L. L. & Wax, M. K. 1998 J Otolaryngol 27:342-347). Studies have demonstrated that good positive predictive value can be achieved by oral cancer tissue staining with toluidine blue (Mashberg, A. & Samit, A. 1995 CA Cancer J Clin 45:328-351). However, extensive experience is required in applying this technique and in interpreting its results. Exfoliative cytology may be a less invasive method for oral cancer detection (Rosin, M. P. et al. 1997 Cancer Res 57:5258-5260). But exfoliated cancer cells tend to correlate with tumor burden, with lower rates of detection seen in those with minimal or early disease. The salivary mRNA biomarkers identified in this study provides a new avenue for OSCC detection. Salivary transcriptome diagnostics meets the demand for a noninvasive diagnostic tool with sufficient predictive power.

For normal individuals, the salivary RNA sources are likely to be from one of the following three sources: salivary glands (parotid, submandibular, sublingual as well as minor glands), gingival crevicular fluids and oral mucosal cells (lining or desquamated). For oral cancer patients, the detected cancer-associated RNA signature is likely to originate from the matched tumor and/or a systemic response (local or distal) that further reflects itself in the whole saliva coming from each of the three major sources (salivary glands, gingival crevicular fluid and oral mucosal cells). It is conceivable that disease-associated RNA can find its way into the oral cavity via the salivary gland or circulation through the gingival crevicular fluid. A good example is the elevated presence of HER-2 proteins in saliva of breast cancer patients (Streckfus, C. et al. 2000 Clin Cancer Res 6:2363-2370). For oral cancer, the local tumor is the source of elevated salivary mRNAs. We have recently selected the most significantly elevated oral cancer tissue transcript, IL8, and confirmed its protein level (by ELISA) is also significantly elevated in saliva of oral cancer patients (St. John, M. A. R. et al. 2004 IL-6 and IL-8: Potential Biomarkers for Oral Cavity and Oropharyngeal SCCA. Archives of Otolaryngology-Head & Neck Surgery, in press). Chen et al. have previously independently demonstrated the elevation of IL8 protein expression in head and neck cancer tissues (Chen, Z. et al. 1999 Clin Cancer Res 5:1369-1379). These data jointly support the concordant alteration of oral cancer associated expression changes in the tumor tissues and saliva, at the mRNA and protein levels.

In addition to IL8, six other cancer-associated genes were identified as being upregulated in saliva from oral cancer patients, such as DUSP, H3F3A, OAZ1, SAT, S100P and IL-1B. DUSP1 gene encodes a dual specificity phosphatase and has been implicated as a mediator of tumor suppressor PTEN signaling pathway (Unoki, M. & Nakamura, Y. 2001 Oncogene 20:4457-4465). The expression of DUSP1 has been shown to decrease in ovarian tumors and a novel single-nucleotide polymorphism (SNP) in the DUSP1 gene has been identified (Suzuki, C. et al. 2001 J Hum Genet 46:155-157). H3F3A mRNA is commonly used as a proliferative marker and its level has been shown to be upregulated in prostate cancers and colon cancers (Bettuzzi, S. et al. 2000 Cancer Res 60:28-34; Torelli, G. et al. 1987 Cancer Res 47:5266-5269). OAZ1 is predicted as a tumor suppressor based on its known inhibitory function to ornithine decarboxylase (ODC) (Tsuji, T. et al. 2001 Oncogene 20:24-33). However, it has been reported that OAZ1 mRNA is upregulated in prostate cancers (Bettuzzi, S. et al. 2000 Cancer Res 60:28-34). Interestingly, the expression of SAT that is also involved in polyamine metabolism has been shown to be significantly higher in prostate cancers (Bettuzzi, S et al. 2000 Cancer Res 60:28-34). S100P is known to be associated with prostate cancer progression and its overexpression is associated with an immortalization of human breast epithelial cells in vitro and early stages of breast cancer development in vivo (Gribenko, A. et al. 1998 Protein Sci 7:211-215; Guerreiro Da Silva, I. D. et al. 2000 Int J Oncol 16:231-240; Mousses, S. et al. 2002 Cancer Res 62:1256-1260; Mackay, A. et al. 2003 Oncogene 22:2680-2688). Recent study shows that differential expression of S100P is associated with pancreatic carcinoma (Logsdon, C. D. et al. 2003 Cancer Res 63:2649-2657; Crnogorac-Jurcevic, T. et al. 2003 J Pathol 201:63-74). The expression of IL-1B is also associated with cancers. The serum level of IL-1B has been shown to be higher in patients with squamous cell carcinoma of oral cavity (Jablonska, E. et al. 1997 Pathol Oncol Res 3:126-129). Also, it has been reported that the level of IL-1B is significantly increased in the ascitic fluid of women with ovarian cancer (Chen, C. K. et al. 1999 J Formos Med Assoc 98:24-30). Genetic polymorphisms of IL-1B have been reported to have potential associations with the risk of diseases, such as gastric cancer and breast cancer (Hamajima, N. & Yuasa, H. 2003 Nippon Koshu Eisei Zasshi 50:194-207; El-Omar, E. M. et al. 2003 Gastroenterology 124:1193-1201).

Saliva is increasingly being used as an investigational aid in the diagnosis of systemic diseases, such as HIV (Malamud, D. 1997 Am J Med 102:9-14), diabetes mellitus (Guven, Y. et al. 1996 J Clin Periodontol 23:879-881), and breast cancer (Streckfus, C. et al. 2000 Clin Cancer Res 6:2363-2370). Most importantly, the concepts, techniques and approach of multiple biomarkers applied in the present Examples could easily be modified to screen and monitor other diseases. For oral cancer, one of the most important applications of the salivary transcriptome diagnostics approach is to detect the cancer conversion of oral premalignant lesions. The overall malignant transformation rates range from 11 to 70.3% (Lee, J. J. et al. 2000 Clin Cancer Res 6:1702-1710; Silverman, S., Jr. & Gorsky, M. 1997 Oral Surg Oral Med Oral Pathol Oral Radiol Endod 84:154-157). Analysis of the DNA content in cells of oral leukoplakia was demonstrated to be useful for predicting the risk of oral cancer (Sudbo, J. et al. 2001 N Engl J Med 344:1270-1278). However, it is still a post-biopsy methodology. We envision that “Salivary Transcriptome Diagnostics”, will provide new opportunities for early diagnostics of oral cancer and other human diseases.

### EXAMPLE 3

### Practical Room Temperature Storage Protocol for Salivary RNA

A practical, user-friendly, room temperature protocol for the optimal preservation of salivary RNA for diagnostic applications was developed. This embodiment of the invention provides salivary RNA of highest quality and quantity for Salivary Transcriptome Diagnostics.

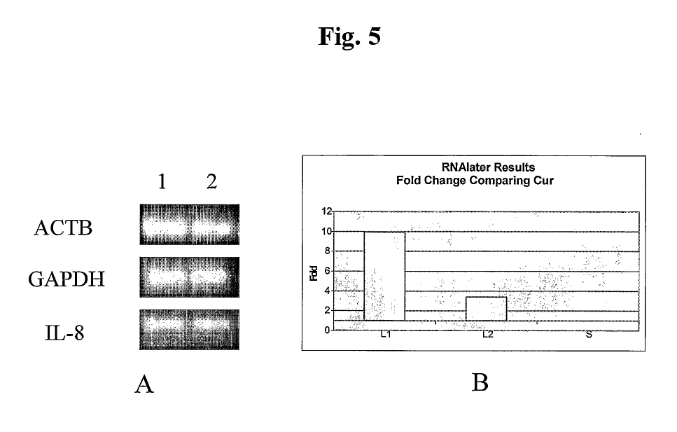

Detection and quantification of human mRNA was performed in RNALater™-treated saliva. Saliva was mixed with 1 or 2 volume(s) of RNAlater™ (Lane 1 or 2). Total RNA from 140 μL saliva supernatant was isolated using Qiagen kit. Aliquots of isolated RNA were treated with DNAse I (Ambion). RT-PCR was used to detect transcripts from three genes, beta-actin (ACTB), glyceraldehyde-3-phosphate dehydrogenase (GAPDH) and interleukin 8 (IL-8) (FIG. 5A). RNA quantification by using Ribogreen® kit (Molecular Probes) showed higher RNA yield from RNAlater™ processed sample other than the Superase-In (Ambion) processed samples (FIG. 5B). Using 1 volume of RNAlater™ (L1) or 2 volumes of RNAlater™ (L2) yielded ˜10-fold and ˜3.3-fold more RNA than the Superase-In (S), respectively. These data were reproduced in samples collected from one same individual in different time-points and in samples collected from 5 different individuals at the same time-point.

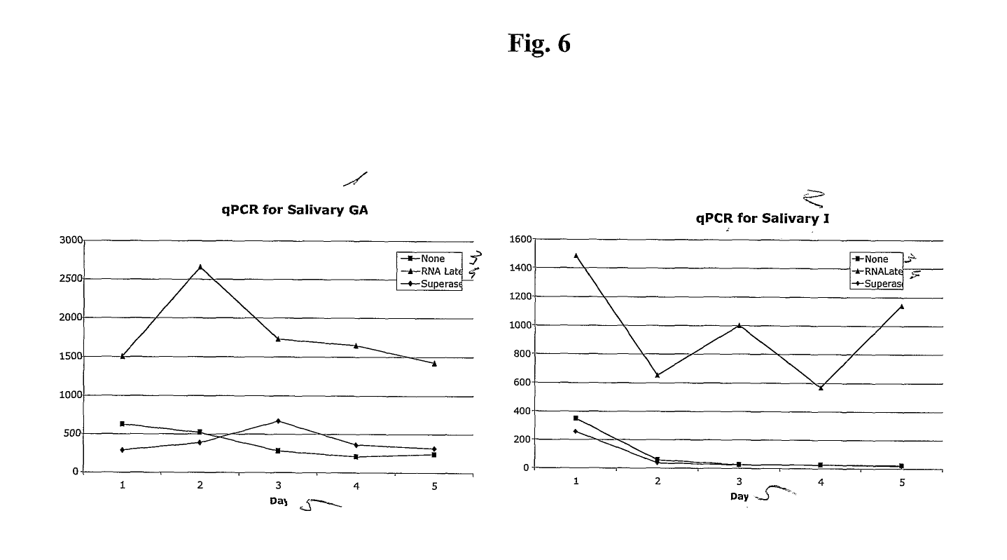

Quantitative PCR (qPCR) was performed to quantify the salivary GAPDH and IL-8. Saliva sample was split into aliquots that were processed with RNAlater™ (1:1 ratio) or Superase-In. Saliva without treatment (None) was used as control. Samples were kept at room temperature for 24 hrs and then stored in 4° C. Total RNA were isolated from 140 μL saliva supernatant in a consecutive 5 days. RT-qPCR were performed from day one to day five to quantify cDNA/RNA encoded by GAPDH and IL-8. Data presented in FIG. 6 indicates that RNAlater™ has a better protective effect on salivary RNA integrity. The term “RNAlater™” is a trademark of Ambion, Inc. (U.S. Pat. No. 6,528,641 and U.S. Pat. No. 6,204,375).

Human salivary mRNA were profiled by using HG U133 plus 2.0 arrays (Affymetrix). The numbers in the Table 7 represent the number of mRNAs that were assigned present by MAS 5.0 and Dchip 1.3.

TABLE 7
Number of present mRNAs on microarrays

| | RNALater ™ | Superase In |
|---|---|---|
| MAS 5.0 | 5,566 | 2,868 |
| Dchip 1.3 | 10,202 | 7,566 |

Data indicates that more mRNAs were recovered by RNAlater™-processed sample than “Superase-In”-processed sample.

This embodiment of the invention is envisioned to be used in any setting where RNA preservation in saliva is desired (e.g., pediatrician's, family doctor's, dentist's, other health care providers' offices, community clinics, home-care kits). The preserved RNA is then shipped to a diagnostic center for specific RNA-based screening or diagnostics as described in Examples 1 and 2. We envision kits for collecting saliva, such as, for example, described in U.S. Pat. Nos. 6,652,481; 6,022,326; 5,393,496; 5,910,122; 5,376,337; 4,019,255; and 4,768,238, combined with RNAlater™-type RNAse inhibiting composition.

Having now fully described the invention, it will be understood to those of ordinary skill in the art that the same can be performed with a wide and equivalent range of conditions, formulations, and other parameters without affecting the scope of the invention or any embodiment thereof. All patents and publications cited herein are fully incorporated by reference hereby in their entirety.

What is claimed is:

1. A method for identifying markers for a human disease state, comprising:
obtaining human saliva sample;
obtaining human mRNAs from said human saliva sample;
amplifying said mRNAs to provide nucleic acid amplification products;
separating said nucleic acid amplification products; and
identifying those mRNAs that are differentially expressed between normal individuals and individuals exhibiting said disease state.

2. The method of claim 1, wherein said disease state is selected from cancers, autoimmune diseases, diabetes and neurological disorders.

3. The method of claim 1, wherein the step of obtaining human mRNAs comprises treating human saliva sample with an RNAse inhibitor.

4. The method of claim 3, wherein said RNAse inhibitor comprises RNAlater™ composition.

5. A method of preserving salivary RNA, comprising:
obtaining a saliva sample;
admixing said sample with RNAlater™ composition.

6. A kit, comprising: a container for collecting saliva, and RNAlater™ composition.
